(19) United States
(12) Patent Application Publication
(10) Pub. No.: US 2009/0111994 A1
(43) Pub. Date: Apr. 30, 2009

(54) Method for The Production of Losartan

(75) Inventors: Yaping Wang, Gansu Province (CN); Yonggang Li, Shanghai (CN); Yulin Li, Gansu Province (CN); Guojun Zheng, Zhejiang (CN); Yi Li, Gansu Province (CN)

Correspondence Address: HEDMAN & COSTIGAN P.C., 1185 AVENUE OF THE AMERICAS, NEW YORK, NY, 10036, US

(73) Assignee: RATIOPHARM GMBH, Ulm (DE)

(21) Appl. No.: 11/883,972

(22) PCT Filed: Feb. 2, 2006
(86) PCT No.: PCT/DE06/00164

§ 371 Date: Sep. 2, 2008

(30) Foreign Application Priority Data
Feb. 3, 2005 (DE) 10 2005 005 047.6

(51) Int. Cl. C07D 257/04

(52) U.S. Cl. 548/252

(57) ABSTRACT

The present invention relates to a novel process for preparing losartan, an imidazole derivative with the chemical name 2-n-butyl-4-chloro-5-hydroxymethyl-1-{[2′-(1H-tetrazol-5-yl)biphenyl-4-]methyl}imidazole, and pharmacologically active salts thereof. The invention further relates to novel intermediates which are suitable for preparing losartan and to novel processes for preparing intermediate compounds which are suitable for preparing losartan.

One aspect of the invention is a process for preparing a compound of the general formula I

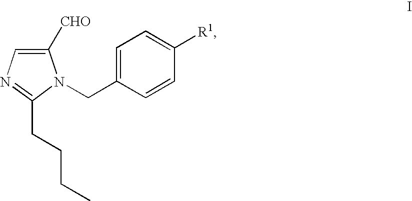

which can form as an intermediate in an inventive preparation of losartan.

The present invention relates to a new method for the production of Losartan, an imidazole derivative with the chemical name 2-n-butyl-4-chloro-5-hydroxymethyl-1-{[2′-(1H-tetrazole-5-yl)biphenyl-4-]methyl}imidazole, as well as its pharmacologically effective salts. Furthermore, the invention relates to new intermediate products, which are suitable for the production of Losartan, as well as new methods for preparing intermediate products, which are suitable for the production of Losartan.

Losartan and efficient and economic ways for its production are of significant interest as Losartan has proven to be a potent active agent for controlling high blood pressure in mammals including humans and disorders resulting therefrom.

Losartan and its production have been described for the first time in EP-A-253 310. The synthesis comprises as essential step an N-alkylation, the reaction of an imidazole with for instance a bromo methyl biphenyl derivate (EP 253 310 B1, p. 213, claim 6).

In EP-A-291 969 there are trityl-protected tetrazole derivatives described, which are suitable for the production of Losartan.

WO 03/093262 relates to the production of Losartan starting from trityl-protected tetrazole derivatives by removal of the protecting group.

The production of Losartan potassium, the usual market form, from Losartan has been described several times (see e.g. EP 324 377 A, page 191, example 316, part D and WO 95/17396, page 18, example 4 and page 24, example 9, step C).

The above-mentioned synthetic processes, however, still seem to need improvement in order to prepare Losartan in an industrial scale, as the overall yield is not satisfactory.

All synthetic routes have in common that first a 1-H-imidazole derivative is prepared, which is then alkylated in position 1. However, with this reaction, there is the possibility that two isomers are formed, depending on which of the two nitrogen atoms is alkylated.

From J. Org. Chem. 1997, 62(24), 8449-8454 (see table 1) there is known the targeted preparation of an imidazole derivative alkylated in position 1 from an N-monosubstituted amidine. The production of suitable precursors for the Losartan synthesis, however, has not been reported.

It is therefore an object to provide new synthetic processes and intermediate products for the production of Losartan and of its pharmacologically effective salts. In particular, it is an object of the invention to provide new synthetic processes and intermediate products for the production of Losartan and of its pharmacologically effective salts by which Losartan is obtainable in a high overall yield.

Furthermore, it is an object of the invention to provide new synthetic methods and intermediate products for the production of Losartan and its pharmacologically effective salts which can be produced also in an industrial scale with little effort concerning the equipment. Furthermore, mostly industrially easily available starting materials should be used, and the use of toxic substances or of substances requiring special labelling should be avoided.

Accordingly, the subject-matter described above has been found.

A central aspect of the invention is the preparation of a compound of the general formula I

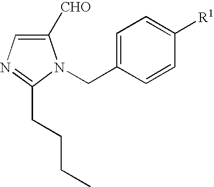

in which R¹ represents a radical R^{1a} or a radical R^{1b}.

R^{1a} is a radical of general formula II,

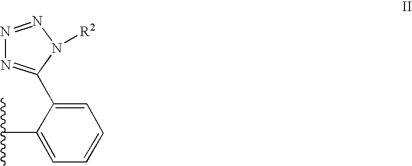

wherein R² represents a tetrazole protecting group.

In general formula II, the “wriggly line” is a symbol for the point of connection, for instance to a compound according to general formula I.

Suitable tetrazole protecting groups in the radical of the above-given general formula II are known from EP-A-291 969 und WO 03/093262 (quod vide the triarylmethyl substituent in the compound of the general formula (II)). Suitable tetrazole protecting groups are in particular triphenylmethyl or tert.-butyl.

Radical R^{1b} in general formula I is a radical which is suitable to bind the phenylene group of the compound of general formula I by a C—C coupling to a further aryl group.

In particular, radical R^{1b} of general formula I is a radical which is capable of coupling the phenylene group of the compound of general formula I by reaction with a radical R³ complimentary thereto, which radical R³ is part of a compound containing a further phenylene unit and having the general formula III,

R³—R⁴ III

wherein R⁴ represents a radical of the general formula II, so as to form a C—C bond between the phenylene group of the compound of general formula I and the phenylene group of the compound of general formula III. The C—C coupling occurs typically with elimination of the radicals R^{1b} and R³.

The compound of general formula I is prepared by reacting a compound of general formula IV

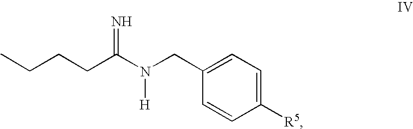

wherein R⁵
in case that R¹ in formula I is a radical R^{1a} represents a radical of general formula II and
in case that R¹ in formula I is a radical R^{1b} has the same meaning as radical R^{1b} in formula I
   with a compound of general formula V,

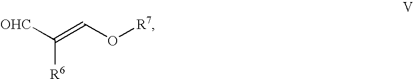

wherein R⁶ represents halogen selected from the group consisting of Cl, Br, I, preferably Br, and R⁷ represents a branched or non-branched C₁-C₆ alkyl group, preferably an isopropyl group.

The above-described reaction (reaction of a compound of general formula IV with a compound of general formula V) is preferably carried out in the presence of a Bronstedt base, in particular a weak Bronstedt base. Suitable Bronstedt bases are alkali metal carbonates or alkali metal hydrogen carbonates, such as sodium carbonate, potassium carbonate or sodium hydrogen carbonate. Preferred is potassium carbonate.

Preferably, the reaction is carried out in a two-phase system, in which one phase is formed from an aqueous solution and the other phase from a solution comprising an organic solvent, not infinitely miscible with water. Examples for suitable solvents are toluene, methylene chloride, chloroform and mixtures thereof.

The reaction of a compound of general formula IV with a compound of general formula V is typically carried out in a molar ratio of 0.5 up to 2:1, relative to the molar amounts of compound of general formula IV to compound of general formula V.

The reaction time is in general 0.1 to 20 hours, preferably 5 to 15 hours.

In the following, radical R^{1b}, which may be contained in compounds of general formula I as well as in compounds of general formula IV (as radical R⁵), is further discussed.

Preferably, radical R^{1b} of the compound of general formula I or radical R⁵ in the compound of general formula IV is a radical which is capable of reaction with radical R³ with formation of a C—C coupling. In particular, radical R^{1b} of the compound of general formula I or radical R⁵ in the compound of general formula IV is a radical which is capable of reaction with radical R³ in a Suzuki, Stille or Grignard reaction.

The terms “Suzuki reaction”, “Stille reaction” or “Grignard reaction” are generally known to the skilled person and are for instance described in Chem. Rev. 2002, 102(5), 1359-1470.

It is particularly preferred that radical R^{1b} in the compound of general formula I or radical R⁵ in the compound of general formula IV has the following meaning:
halogen, in particular bromine,
a radical of general formula VI

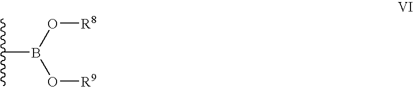

wherein R⁸ and R⁹ represent hydrogen, a C₁ to C₆ alkyl group or together a C₁ to C₆ alkandiyl group,
a trialkyl tin radical, wherein “alkyl” preferably represents a C₁ to C₁₂, in particular a C₁ to C₆ alkyl radical, or
if in the process a compound of general formula I with radical R^{1b} is used, a magnesium(II) halide radical,
   and wherein
if R^{1b} or R⁵ represents halogen, R³ represents a radical of general formula VI, a trialkyl tin radical or, if in the process a compound of general formula I with radical R^{1b} is used, a magnesium(II) halide radical, and vice versa.

In the radical of general formula VI, radicals R⁸ and R⁹ preferably together represent 2,3-dimethylbutane-2,3-diyl.

It is furthermore especially preferred that radical R³ in general formula III is selected from the group comprising the following radicals:
halogen, preferably bromine
a radical of general formula VI
a trialkyl tin radical, wherein “alkyl” preferably represents a C₁ to C₁₂, in particular a C₁ to C₆ alkyl radical, or
a magnesium(II) halide radical, preferably a magnesium(II)bromide radical,
   however, with the proviso that
radical R^{1b} and radical R⁵ on the one hand and radical R³ on the other hand are selected such that radicals R^{1b} and R⁵ on the one hand and radical R³ on the other hand are complimentary radicals, i.e. that they form complimentary pairs which can react with each other in the desired way in a coupling reaction.

Thus, the radicals R^{1b} and R³ or R⁵ and R³ are to be selected such that they form complimentary pairs. Preferred complimentary pairs are:
a) halogen and a radical of general formula VI, because they can react with each other, for instance in a Suzuki reaction,
b) halogen and a trialkyl tin radical, wherein “alkyl” preferably represents a C₁ to C₁₂, in particular C₁ to C₆ alkyl radical, because they can react with each other for instance in a Stille reaction, and
c) halogen and a magnesium(II) halide radical; because they can react with each other in a Grignard reaction.

Halogen preferably represents bromine, and the magnesium(II) halide radical preferably represents a magnesium(II)bromide radical.

In the Suzuki, Stille or Grignard reactions, which are employed in the inventive preparation method, it is advantageous to employ one or more catalysts. The catalysts may comprise one or more transition metals, in particular manganese, chromium, iron, cobalt, nickel or palladium. Preferably employed compounds are selected among MnCl₂, CrCl₃, FeCl₂, Fe(acac)₃, FeCl₃, Fe(salen)Cl, NiCl₂(PPh₃)₂, COCl₂(dppe), COCl₂(dpph), Co(acac)₂, COCl₂(dppb), PdCl₂(PPh₃)₂ and Pd(PPh₃)₄.

Advantageously, the catalysts are used together with an activator and/or stabilizer. The activator transfers the metal atoms of the catalysts to oxidation state 0, and the stabilizer stabilizes the metal atoms of the catalysts in the oxidation state 0. Examples for such activators are zinc (preferably in the form of zinc powder), sodium borohydride, lithium aluminium hydride or organic compounds of aluminium, magnesium or lithium (preferably butyl lithium or DIBAH). Examples for such stabilizers are Lewis bases, preferably phosphanes, particularly preferred triaryl phosphanes and trialkyl phosphanes, in particular triphenyl phosphanes.

In the Suzuki reaction, it is in particular advantageous to use a palladium catalyst, such as Pd(PPh₃)₄ as a catalyst. The reaction is preferably carried out in the presence of a weak Bronstedt base, such as an alkali metal carbonate. It is advantageous to carry out the reaction in a two-phase system, in which one phase is formed from an aqueous solution and the other phase is formed from a solution comprising an organic solvent not infinitely miscible with water, such as benzene, toluene, xylene, methylene chloride or chloroform.

In the Stille reaction, it is in particular advantageous to employ a palladium catalyst, such as Pd(PPh₃)₄ or PdCl₂(PPh₃)₂ as a catalyst. The reaction is preferably carried out at elevated temperature, preferably at temperatures between 50° C. and the boiling point of the solvent. It is advantageous to carry out the reaction in the presence of a co-catalyst, such as CuI (copper iodide) or CuO (copper oxide). The reaction is preferably carried out in an inert solvent, such as for example toluene, xylene, dimethoxy ethane, dimethyl formamide, tetrahydrofurane, or dioxane.

In the Grignard reaction, it is particularly advantageous to use a palladium catalyst such as Pd(PPh₃)₄, PdCl₂(PPh₃)₂ or NiCl₂(PPh₃)₂ as a catalyst. The reaction is preferably carried out in polar, aprotic solvents, such as tetrahydrofurane, diethylether or dioxane.

Compounds of general formula I are in general prepared by reacting compounds of general formula (IV) with compounds of general formula (V) as described above. Depending on the kind of the radicals R¹ and R⁵, different synthetic routes are preferred.

Within the framework of the invention, three preferred synthetic routes are further described, which are referred to as synthetic route A, synthetic route B and synthetic route C.

Synthetic route A describes the case that R⁵ in compounds of general formula IV represents a radical of general formula II. (This means that radical R⁵ in formula IV corresponds to radical R^{1a} in formula I.)

In general, the preparation of the compound of general formula IV, wherein R⁵ represents a radical of general formula II, is carried out by reacting a compound of general formula VII

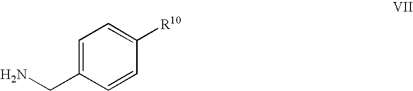

wherein R¹⁰ represents a radical of formula II,
with a compound of general formula VIII

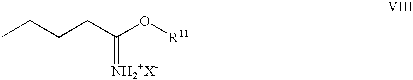

wherein R¹¹ represents a C₁ to C₁₂ alkyl radical and X represents the anion of an inorganic acid,
preferably in the presence of a Bronstedt base.

The reaction is typically carried out in a suitable inert solvent, for instance an aliphatic alcohol, such as ethanol. As a Bronstedt base, there is suitably used for instance a tertiary aliphatic amine, such as triethyl amine, for neutralisation.

The reaction is carried out in a molar ratio of 0.3 to 3:1, relative to the molar amount of compounds of general formula VII in relation to the compound of general formula VIII. The reaction time is in general 0.1 to 20 hours, preferably 5 to 15 hours.

It is particularly advantageous to carry out the preparation of the compounds of general formula VII by:
I. providing a compound of general formula IX

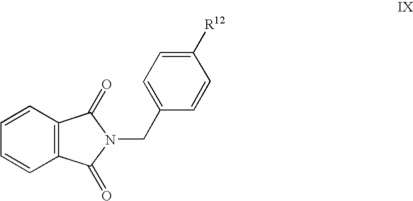

wherein R¹² is a radical of general formula II and
II. preparing from the compound of general formula IX under conditions which are usual for a Gabriel reaction, the compound of general formula VII. For this end, the compound of general formula IX is reacted for instance with hydrazine hydrate in alcoholic solution.

The preparation of a compound of general formula IX is for instance known from Bioorganic & Medicinal Chemistry Letters 1993, 3(4), 757-760 (referred to therein as compound 20).

The preparation of a compound of general formula VIII is for instance known from J. Org. Chem. 1999, 64(22), 8084-8089.

Synthetic route B describes the case that R⁵ in compounds of general formula IV represents a halogen atom, in particular bromine. (This means that radical R⁵ in formula IV corresponds to radical R^{1b} in formula I.)

For preparing a compound of general formula IV, wherein R⁵ represents halogen, it is preferred to react a benzyl amine derivative (i.e. a compound which corresponds to general formula VII) substituted in para position with a halogen atom with a compound of general formula VIII, preferably in the presence of a Bronstedt base. For the conditions of this reaction the same applies as for the preparation of the compound of general formula IV wherein R⁵ represents a radical of general formula II.

The benzyl amine derivative substituted in para position with a halogen atom is prepared in a particularly easy way by a Gabriel reaction under the typical conditions for a Gabriel reaction with phthalimide from a benzyl halide substituted in para position with a halogen atom, preferably bromine, in particular para bromo benzyl bromide.

In synthetic route B, the reaction of the compound of general formula IV with the compound of general formula V leads to a compound of general formula I, wherein radical R¹ is a radical R^{1b}.

According to a preferred embodiment, the obtained compound of general formula I with a radical R^{1b} is transformed by one of the above-described C—C coupling reactions to a compound of general formula I with a radical R^{1a}.

The preparation of a compound of general formula I with a radical R^{1a} is preferably carried out by reacting a compound of general formula I, wherein R^{1b} represents halogen, preferably bromide, with a compound of general formula III, wherein R³ represents a radical of general formula VI, a trialkyl tin radical or a magnesium(II) halide radical, under conditions which are typical for a Suzuki, Stille or Grignard reaction.

Synthetic route C describes the case that R⁵ in compounds of general formula IV represents a radical of general formula VI. (This means that R⁵ in formula IV corresponds to R^{1b} in formula I.)

The preparation of a compound of general formula IV, wherein R⁵ represents a radical of general formula VI, is preferably carried out by reacting a benzyl amine derivative substituted in para position with a radical R⁵ of general formula VI with a compound of general formula VIII, preferably in the presence of a Bronstedt base.

The benzyl amine derivative substituted in para position with a radical R⁵ of the general formula VI is in general prepared in a Gabriel reaction under the conditions typical for a Gabriel reaction with phthalimide from a benzyl halide substituted in para position with a radical R⁵ of general formula VI, preferably a benzyl bromide substituted with a radical R⁵ of general formula VI.

In synthetic route C, the reaction of the compound of general formula IV with the compound of general formula V leads to a compound of general formula I, wherein radical R¹ is a radical R^{1b}.

According to a preferred embodiment, the obtained compound of general formula I with radical R^{1b} is transformed via one of the above-described C—C coupling reactions into a compound of general formula I with a radical R^{1a}.

The compound of general formula I wherein R¹ represents a radical of general formula II is prepared according to an especially preferred method, by reacting a compound of general formula I wherein R^{1b} represents a radical of general formula VI with a compound of general formula III wherein R³ represents halogen, preferably bromine, under conditions as are typical for a Suzuki reaction.

In case that in general formula IV R⁵ is a trialkyl tin or MgHal radical, the course of the reaction as in synthetic route C is also preferred.

A further aspect of the invention is the preparation of an imidazole derivative which is substituted at at least one carbon atom of the imidazole ring with chlorine (imidazole derivative A) by reacting imidazole or an imidazole derivative carrying at at least one carbon atom of the imidazole ring a hydrogen atom (imidazole derivative B), with CeCl₃ and an alkali metal salt of a hypohalous acid.

Preferably, as imidazole derivative (A) a compound is prepared which is substituted at the carbon atom of the imidazole ring in the 4 or 5 position or at both of these positions with chlorine, and as imidazole derivative (B) a compound is employed still carrying at the carbon atom of the imidazole ring in the 4 or 5 position or at both of these positions a hydrogen atom.

This chlorination method is particularly suitable for the preparation of a Losartan derivative wherein the hydrogen atom of the tetrazole group is replaced by a tetrazole protecting group, wherein as imidazole derivative (B) the compound of general formula IX is employed.

Preferably, CeCl₃ and the alkali metal salt of the hypohalous acid are employed in stoechiometric amounts or in an excess. As the alkali metal salt of the hypohalous acid, the potassium or sodium salt is advantageously employed. Preferably, as the alkali metal salt of the hypohalous acid, an alkali metal salt of hypochlorous acid is employed.

The chlorination reaction according to the invention is typically carried out in a two-phase system, in which one phase is formed from an aqueous solution and the other phase is formed from a solution comprising an organic solvent not infinitely miscible with water, such as methylene chloride, chloroform or toluene. Starting from a compound of general formula I with a radical R^{1a}, Losartan or one of its pharmacologically acceptable salts can be prepared in a particularly simple manner by
a) preparing in a step (a) staring from a compound of general formula I with a radical R^{1a} the compound of general formula XI

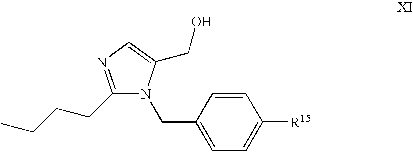

wherein R¹⁵ represents a radical of general formula II, by reducing the formyl group, with which the imidazole group is substituted, to a hydroxy methyl group,
b) replacing in a step (b) the sole remaining hydrogen atom in the imidazole group of the compound prepared according to step (a) by chlorine and
(c) removing in a step (c) in the compound prepared according to step (b) the tetrazole protecting group and optionally
(d) preparing from Losartan one of its pharmacologically acceptable salts, such as the potassium salt.

The reduction of the formyl group in step (a) can be prepared in the usual manner. Preferably, the reduction of the formyl group in step (a) is carried out with sodium borohydride or lithium aluminium hydride.

The chlorination in step (b) can be carried out in the usual manner. Preferably, step (b) is carried out by employing the above-described chlorination method, i.e. by using CeCl₃.

Step (c) is usually carried out as described in WO 03/093262. The removal of the especially preferred triphenyl methyl protecting group can be achieved for instance by treating a solution of the compound prepared according to step (b) with a diluted mineral acid, preferably hydrochloric acid.

The preparation of a pharmacologically acceptable salt of Losartan, for instance Losartan potassium (step (d)), is carried out as for instance described in EP 324 377 A, page 191, example 316, part D and WO 95/17396, page 18, example 4 and page 24, example 9, step C.

Below the synthetic routes A, B and C described above are further explained.

The compounds used and/or being formed in the respective synthetic routes are referred to by Arabic numerals. With respect to the compounds described in the reaction schemes, the following applies:
4 corresponds to a compound of general formula I with a radical R¹ of general formula II (i.e. R¹ is a radical R^{1a}),
15 and 21 correspond to compounds of general formula I with a radical R^{1b}
13, 23 correspond to compounds of general formula III,
3, 14 correspond to compounds of general formula IV,
8 corresponds to a compound of general formula V,
21 corresponds to a compound of general formula IV with a radical R⁵ of general formula VI,
1 corresponds to a compound of general formula VII,
2 corresponds to a compound of general formula VIII, and
9, 10 correspond to compounds of general formula IX.

At the same time, the compounds are preferred working examples of the compound groups defined by the respective general formulae.

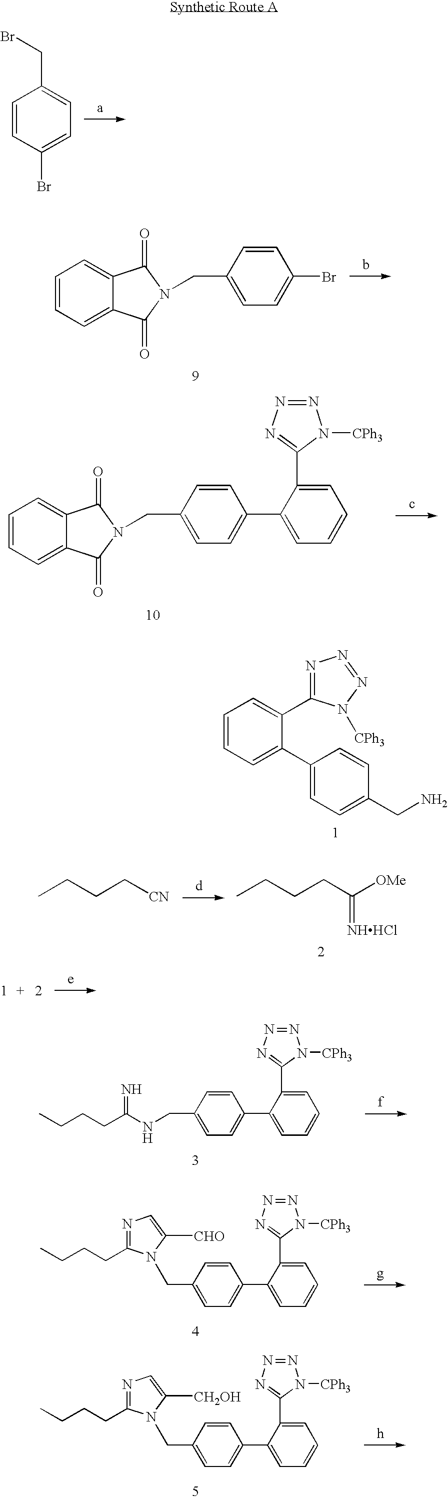

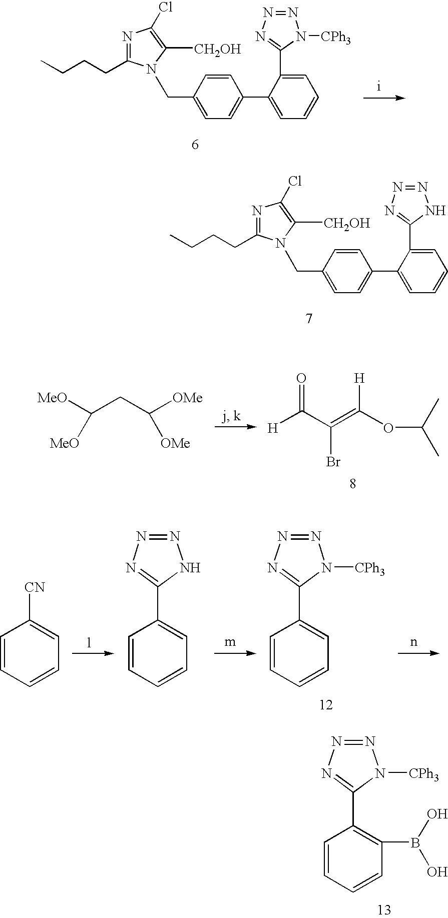

The reactions a) to n) are carried out in general under usual reaction conditions, preferably in the presence of the following reagents:
a) phthalimide
b) 13
d) CH₃OH
e) base
f) 8
g) hydrogenation agent
h) chlorination agent

According to a specially preferred embodiment, the following reagents and reaction conditions can be applied:
a) phthalimide, K₂CO₃/DMSO;
b) 13, Pd(PPh₃)₄, Na₂CO₃, toluene-H₂O, 80° C.;
c) hydrazine hydrate, CH₃OH/CH₂Cl₂;
d) CH₃OH/HCl;
e) NEt₃/EtOH;
f) 8, K₂CO₃/CHCl₃—H₂O;
g) NaBH₄/CH₃OH;
h) CeCl₃.7H₂O, NaClO/CH₂Cl₂—H₂O;
i) 2N HCl, CH₃OH-THF;
j) concentrated HCl, Br₂;
k) PPTS, isopropanol;
l) NaN₃, NH₄Cl, LiCl, DMF, 100° C.;
m) Ph₃CCl, Et₃N/CH₂Cl₂;
n) BuLi, −20° C. to −5° C., then B(OMe)₃, −20° C. to room temperature

According to a preferred embodiment, the compounds 1 and 2 are reacted to compound 4 without isolating compound 3.

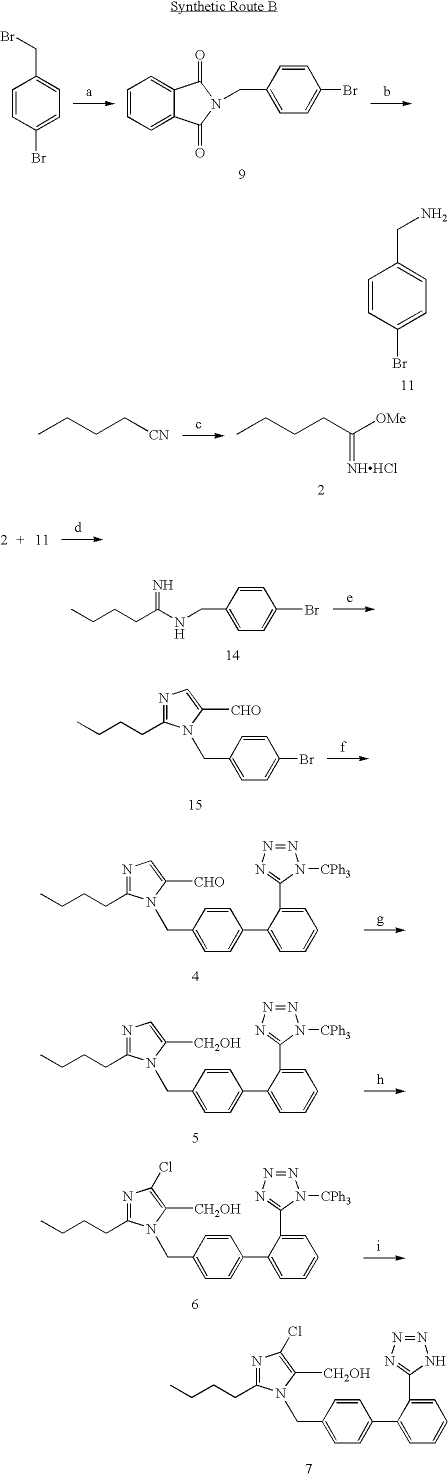

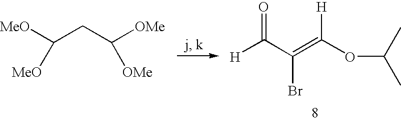

The reactions a) to k) are in general carried out under usual reaction conditions, preferably in the presence of the following reagents:
a) phthalimide
c) CH₃OH
d) base
e) 8
f) 13
g) hydrogenation agent
h) chlorination agent

According to a particularly preferred embodiment, the following reagents and reaction conditions can be used:
a) phthalimide, K₂CO₃/DMSO;
b) hydrazine hydrate, CH₃CH₂OH/H₂O;
c) CH₃OH/HCl;
d) NEt₃/EtOH;
e) 8, K₂CO₃/CHCl₃—H₂O;
f) 13, Pd(PPh₃)₄, Na₂CO₃, toluene-H₂O, 80° C.;
g) NaBH₄/CH₃OH;
h) CeCl₃.7H₂O, NaClO/CH₂Cl₂—H₂O;
i) 2N HCl, CH₃OH-THF;
j) concentrated HCl, Br₂;
k) PPTS, isopropanol;

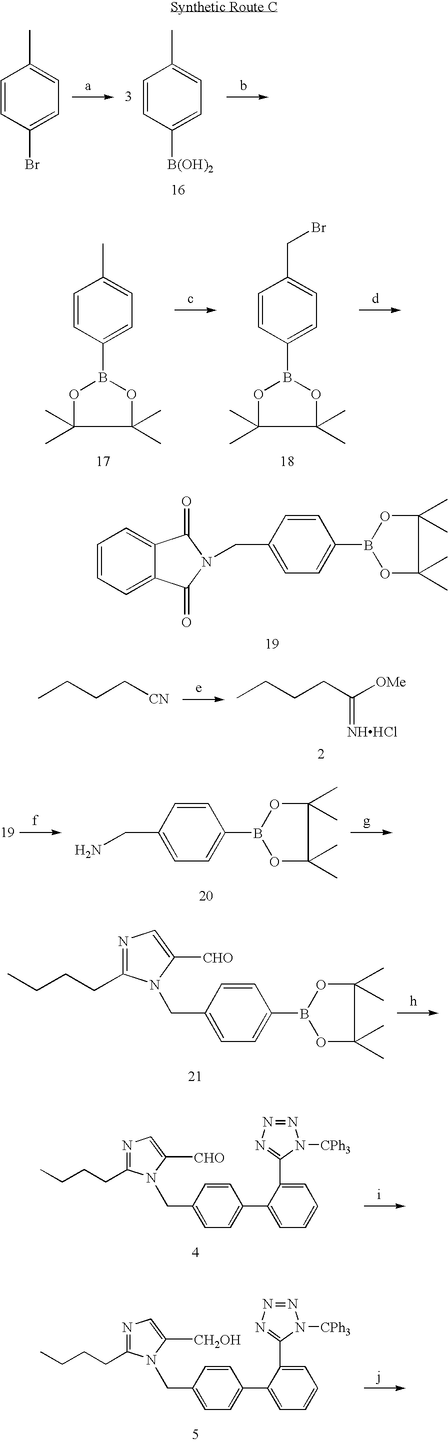

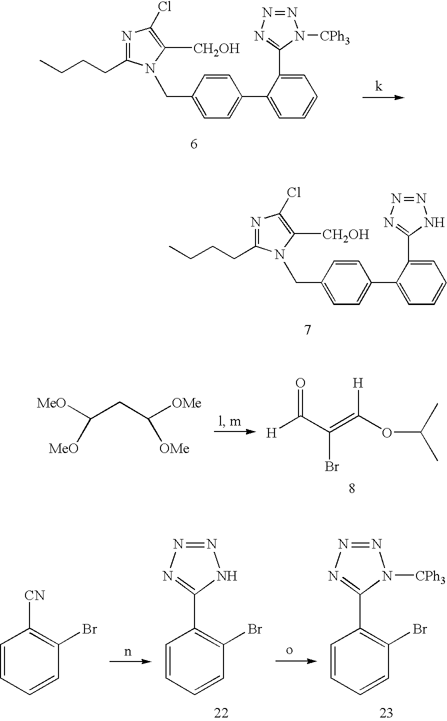

The reactions a) to o) are carried out in general under usual reaction conditions, preferably in the presence of the following reagents:
d) phthalimide
e) CH₃OH
g) 2, base, then 8
h) 13
i) hydrogenation agent
j) chlorination agent

According to a particularly preferred embodiment, the following reagents and reaction conditions can be used:

a) Mg, I₂, THF, reflux 1 h, then −78° C., B(OMe)₃;

b) pinacole, cyclohexane, reflux to remove water;

c) NBS, cyclohexane, reflux;

d) phthalimide, K₂CO₃/acetone, reflux;

e) CH₃OH/HCl;

f) hydrazine hydrate, CH₃CH₂OH, reflux;

g) 2, NEt₃/CH₃OH, then K₂CO₃, 8;

h) 13, Pd(PPh₃)₄, K₂CO₃, toluene-H₂O, 80° C.;

i) NaBH₄/CH₃OH;

j) CeCl₃.7H₂O, NaClO/CH₂Cl₂—H₂O;

k) 2N HCl, CH₃OH-THF;

l) concentrated HCl, Br₂;

m) PPTS, isopropranol;

n) NaN₃, NH₄Cl, LiCl, DMF, 100° C.;

o) Ph₃CCl, Et₃N/CH₂Cl₂.

### EXPERIMENTAL PART

In the experimental part, the preparation of the compounds occurring in the synthetic routes A, B or C is further described.

### Description of Experiments

### Equipment and Reagents

All dry solvents (CH₂Cl₂, THF, Et₂O, benzene, toluene, DMF, MeCN) were dried according to standard methods, i.e. by removing water and oxygen and distillation prior to use. The reactions were carried out as far as necessary under an inert gas atmosphere (N₂ or Ar) and were monitored by TLC. The solvents for the extraction were for example diethyl ether, ethyl acetate or chloroform. The extracts were, if not stated otherwise, dried, for instance with anhydrous MgSO₄. The reaction products were, as far as necessary, purified, for instance by column chromatography using for example petrol ether (60-90° C.)/ethyl acetate and chloroform/methanol as eluent. If plates of type GF₂₅₄ were used for TLC, the detection agent iodine or an ethanolic solution of phosphor molybdanic acid were used. The silicagel for the chromatography (200-300 mesh) and TLC (GF₂₅₄) were prepared by Qingdao Sea Chemical Factory and Yantan Chemical Factory. All solvents and reagents were of analytical or chemical purity.

The melting points were determined with an XT₄-100x micro-melting point tester. Nicolet AVATAR 360 FT-IR and Nicolet NEXUS 670 FT-IR spectrometers were used for recording infra red spectra using KBr tablets or PE films. Mercury-300 (Varian) and AM-400 (Bruker) spectrometers were used for NMR measurements with SiMe₄ as internal standard and CDCl₃ as solvent, as far as nothing else is reported. LRMS were determined with a HP-5988 mass spectrometer using EI at 70 eV, unless otherwise reported. HRMS were measured using a Bruker Daltonics APEX II 47e FT-ICR mass spectrometer.

### Preparation of Compound 9

Phthalimide (11 g, 75.6 mmol) was dissolved in 80 ml of DMSO under argon protecting atmosphere. After addition of K₂CO₃ (20 g, 144 mmol) the reaction mixture was heated for two hours at 120° C. Thereafter, the reaction mixture was cooled to about 50° C., and p-bromo benzyl bromide (18 g, 72 mmol) was added. After further ten hours of stirring, 100 ml H₂O were added. The colourless precipitate was filtered off, washed and dried, which led to compound 9 (18.6 g). The yield was 82%.

¹H NMR (CDCl₃, 300 MHz): δ 4.77 (s, 2H, NCH₂), 7.29 (d, J=8.4 Hz, 2H, ArH), 7.41 (d, J=8.4 Hz, 2H, ArH), 7.67-7.70 (m, 2H, ArH), 7.80-7.83 (m, 2H, ArH); ¹³C NMR (CDCl₃, 75 MHz): δ 40.9, 121.8, 123.3 (2C), 130.3 (2C), 131.7 (2C), 131.9 (2C), 134.0 (2C), 135.2, 167.8 (2C); MS (FAB): M⁺=315, found: 316 (M⁺+1), 318 (M⁺+3); IR (film, cm⁻¹) νₘₐₓ=3460, 3100, 3045, 2938, 1771, 1702, 1612, 1486, 1464, 1430, 1399, 1332, 1298, 1173, 1077, 1010, 957, 935, 845, 796, 731, 713, 528.

### Preparation of Compound 10

Compound 9 (1.45 g, 4.6 mmol), compound 13 (3.4 g, 1.2 equivalents) and Na₂CO₃ (1.46 g, 3 equivalents) were dissolved in a mixture of 20 ml toluene/H₂O (7:3). Thereafter, the system was purged three times with argon, and Pd(PPh₃)₄ (266 mg, 0.05 equivalents) was added. The reaction mixture was heated for thirteen hours at 80° C. and thereafter extracted with ethyl acetate. The organic phases were combined, and the solid was purified by column chromatography which led to compound 10 as a white solid (2.43 g). The yield was 86%.

¹H NMR (CDCl₃, 300 MHz): δ 4.73 (s, 2H), 6.87-6.90 (m, 6H), 7.06 (d, J=8.1 Hz, 2H), 7.18-7.33 (m, 12H), 7.42-7.46 (m, 2H), 7.67-7.70 (m, 2H), 7.82-7.84 (m, 2H), 7.92-7.95 (m, 1H); ¹³C NMR (CDCl₃, 75 MHz): δ 41.2, 82.8, 123.3, 126.2, 127.6, 128.2, 129.5, 129.8, 130.2, 130.7, 132.1, 133.9, 134.8, 140.7, 141.2, 141.7, 163.9, 167.9; MS (FAB): M⁺=623, found: 662 (M⁺+K); IR (film, cm⁻¹) νₘₐₓ=3467, 3061, 3032, 2249, 1770, 1714, 1603, 1492, 1469, 1446, 1429, 1393, 1349, 1187, 1159, 1087, 1031, 1004, 937, 909, 880, 733, 700, 633, 406.

### Preparation of Compound 1

Compound 10 (37.3 g, 60 mmol) was dissolved in a mixture of 200 ml of methanol and 300 ml of CH₂Cl₂. Hydrazine hydrate (600 mmol, 10 equivalents) was added, and the reaction mixture was stirred for ten hours at room temperature. Thereafter, a filtration was carried out and the filtrate was diluted with CHCl₃ and washed with water. The organic phase was dried over MgSO₄ and concentrated, which led to compound 1 as a yellowish solid (23.7 g). The yield was 80%.

¹ H NMR (CDCl₃, 200 MHz): δ 3.80 (s, 2H), 4.52 (s, br, 2H), 6.89-6.92 (m, 6H), 7.04-7.14 (m, 4H), 7.24-7.31 (m, 10H), 7.43 (s, br, 2H), 7.90-7.93 (m, 1H); MS (FAB): M⁺=493, found: 494 (M⁺+1), 516 (M⁺+Na).

### Preparation of Compound 2

HCl gas was inserted into a solution of 8.3 g (100 mmol) valeronitrile in 8 ml methanol under ice bath cooling. The temperature was always kept below 10° C. After two hours, the reaction was finished, and compound 2 was obtained as a white solid.

¹H NMR (CDCl₃, 300 MHz): δ 0.84 (t, J=7.2 Hz, 3H, CH₃), 1.26-1.34 (m, 2H, CH₂), 1.57-1.67 (m, 2H, CH₂), 2.68 (t, J=7.5Hz, 2H, CH₂), 4.19 (s, 3H, OMe), 7.43 (s, 1H, NH), 11.12 (s, br, 1H, HCl), ¹³C NMR (CDCl₃, 75 MHz): δ 13.2, 21.7, 27.3, 32.5, 60.4, 180.6.

### Preparation of Compound 8

Concentrated HCl (3.6 ml) was added dropwise to a mixture of 1,1,3,3-tetramethoxypropane (6.5 g, 40 mmol) and water (64 ml). The reaction mixture was homogenised by stirring, cooled to 0° C. and added dropwise with bromine (2.1 ml, 40 mmol). Stirring was continued for further 10 minutes and thereafter a large proportion of the water was removed under vacuum at 70° C. After cooling again to 0° C., a filtration was carried out. The solid obtained in this way was dried and thereafter dissolved in a mixture of 65 ml cyclohexane and 10 ml isopropanol. A catalytic amount of PPTS was added. The reaction mixture was heated for 90 minutes under reflux, and the formed water was removed. The remaining solvent was removed under vacuum. Compound 8 remained as a yellow oil. The purity was larger than 95% so that compound 8 could be used without further purification. The yield was 65%.

¹H NMR (CDCl₃, 300 MHz): δ 1.38 (d, J=6 Hz, 6H, 2CH₃), 4.45-4.50 (m, 1H, CH), 7.70 (s, 1H, CH═), 9.07 (s, 1H, CHO); ¹³C NMR (CDCl₃, 75 MHz): δ 22.3 (2C), 80.6, 105.1, 166.2, 184.0; MS (EI) m/z (%):194 (M⁺, 5), 192 (M⁺, 5), 166 (2), 152 (95), 150 (100), 121 (13), 93 (16), 71 (30), 43 (59).

### Preparation of Compound 3

Compound 2 (6.4 g, 40 mmol) was dissolved in 75 ml of absolute ethanol and 20 ml of NEt₃. Afterwards, compound 1 (10 g, 20 mmol) was added at 10° C., and the reaction mixture was stirred for five hours, which led to a solution, which was stirred for a further eight hours at room temperature. Thereafter it was diluted with chloroform and washed with water. The organic phase was separated, dried and concentrated at a temperature of below 45° C. Compound 3 was obtained as an oil. The yield was 80%.

¹H NMR (CDCl₃, 300 MHz): δ 0.67-0.80 (m, 3H, CH₃), 1.09-1.24 (m, 2H, CH₂), 1.58 (s, br, 2H, CH₂), 2.51 (s, br, 2H, CH₂), 4, 53 (s, br, 2H, NCH₂), 6.89-7.08 (m, 8H, ArH), 7.21-7.44 (m, 14H, ArH), 7.87 (d, J=3.9Hz, 1H, ArH); ¹³C NMR (CDCl₃, 75 MHz): δ 13.3, 21.6, 28.0, 32.3, 45.4, 82.9, 125.5, 126.0, 127.5, 128.2, 129.2, 129.8, 133.3, 140.3, 140.7, 141.0, 163.9, 167.7; MS (FAB): M⁺=576, found: 577 (M⁺+1); IR (film, cm⁻¹) νₘₐₓ=3222, 3057, 3030, 2962, 2933, 2210, 1677, 1636, 1531, 1493, 1449, 1190, 1004, 910, 732, 700, 639.

### Preparation of Compound 4

Method A-1 (with Isolation of Compound 3):

Compound 3 (8 g, 13.9 mmol) was dissolved in 60 ml of chloroform and 7.5 ml of water. After addition of K₂CO₃ (2.69 g, 19.5 mmol) and compound 8 (3.73 g, 19.5 mmol) the reaction mixture was stirred for twelve hours at room temperature. Thereafter, an extraction with chloroform was carried out and the organic phase was concentrated. The thus obtained raw product was purified by column chromatography, which led to compound 4 as a white solid (3.67 g). The yield was 42%.

### Method A-2 (Without Isolation of Compound 3):

Compound 2 (4.5 g, 1.5 equivalents, 30 mmol) was dissolved in a mixture of 50 ml absolute ethanol and NEt₃ (8.3 ml, 3 equivalents) at 0° C., and compound 1 (10 g, 1 equivalent, 20 mmol) was added. The reaction mixture was stirred for about five hours to obtain a clear solution and furthermore for sixteen hours at room temperature. Thereafter, K₂CO₃ (4.14 g, 30 mmol, 1.5 equivalents) and compound 8 (4.6 g, 1.2 equivalents, 24 mmol) were added, and stirring was continued for twelve hours at room temperature. The reaction mixture was extracted with CHCl₃, and the organic phase was concentrated. The obtained solid was purified by column chromatography, which led to compound 4 as a white solid.

### Method A-3 (Without Isolation of Compound 3):

Compound 1 (563 g) was dissolved in a mixture of 3.6 l of absolute ethanol and 1.2 l of triethyl amine. The reaction mixture was cooled to 0° C., and compound 2 (350 g) was slowly added. Stirring was carried out for one hour at 0° C., and thereafter the reaction mixture was diluted with chloroform and water. The organic phase was separated, and the aqueous phase was again extracted with chloroform. To the combined organic phases, K₂CO₃ (180 g), 560 ml water and 330 g compound 8 were added at room temperature. Thereafter, stirring was carried out at room temperature over night, and the reaction mixture was diluted with chloroform and water. The organic phase was separated, and the aqueous phase was again extracted with chloroform. The combined organic phases were dried with MgSO₄, filtered and concentrated. The radical thus obtained was recrystallised from ethyl acetate, which led to compound 4 (530 g). The yield was 74%.

### Method B:

Compound 15 (1.47 g, 4.6 mmol), compound 13 (3.4 g, 1.2 equivalents) und Na₂CO₃ (1.46 g, 3 equivalents) were dissolved in 20 ml of a mixture of toluene and water (7:3). Thereafter, the system was three times purged with argon, and Pd(PPh₃)₄ (266 mg, 0.05 equivalents) was added. The reaction mixture was heated for ten hours at 80° C. and then extracted with ethyl acetate. The organic phases were concentrated and the radical was purified by column chromatography, which led to compound 4 as a solid (2.1 g). The yield was 74%.

### Method C:

Compound 21 (40 mg, 0.11 mmol), compound 23 (151 mg, 0.33 mmol) and K₂CO₃ (45 mg, 0.33 mmol) were dissolved in 3 ml of a mixture of toluene and water (7:3). Thereafter, the system was purged three times with argon, and Pd(PPh₃)₄ (6 mg, 0.05 equivalents) was added. The reaction mixture was heated for ten hours at 80° C. and then extracted with ethyl acetate. The organic phases were concentrated, and the radical was purified by column chromatography, which led to compound 4 as a solid (48 mg). The yield was 72%.

¹H NMR (CDCl₃, 400 MHz,): δ 0.86 (t, J=7.2 Hz, 3H, CH₃), 1.25-1.32 (m, 2H, CH₂), 1.64-1.68 (m, 2H, CH₂), 2.55 (t, J=7.6 Hz, 2H, CH₂), 5.48 (s, 2H, NCH₂), 6.82 (d, J=8.4 Hz, 2H, ArH), 6.91-6.93 (m, 6H, ArH), 7.09 (d, J=8.4 Hz, 2H, ArH), 7.23-7.27 (m, 6H, ArH), 7.31-7.35 (m, 4H, ArH ), 7.41-7.49 (m, 2H, ArH), 7.78 (s, 1H, CH═), 7.91 (dd, J=1.2 Hz, 7.2 Hz, ArH), 9.64 (s, 1H, CHO); ¹³C NMR (CDCl₃, 100 MHz,): δ 13.7, 22.3, 26.4, 29.2, 47.8, 82.8, 125.9, 126.2, 127.6, 127.8, 127.9, 128.2, 129.7, 129.9, 130.1, 130.2, 130.7, 131.3, 134.7, 140.7, 141.2, 141.4, 143.6, 156.7, 163.8, 178.5; MS (FAB): M⁺=628, found: 629 (M⁺+1), 651 (M⁺+Na); IR (film, cm⁻¹) νₘₐₓ=3060, 3031, 2959, 2932, 2868, 1670, 1619, 1597, 1532, 1466, 1446, 1187, 1160, 1030, 1003, 909, 880, 824, 762, 733, 701, 640.

### Preparation of Compound 5

Compound 4 (6.3 g, 10 mmol) was suspended in 30 ml of methanol and 3 ml of CHCl₃ were added for complete dissolution. The reaction mixture was cooled in an ice bath, and NaBH₄ (760 mg, 20 mmol) was added. After one hour of stirring, the mixture was extracted with CHCl₃. The organic phase was concentrated, which led to compound 5 (6 g) as an oil. The yield was 95%.

¹H NMR (CDCl₃, 300 MHz): δ 0.85 (t, J=7.5 Hz, 3H, CH₃), 1.27-1.32 (m, 2H, CH₂), 1.61-1.66 (m, 2H, CH₂), 2.50 (t, J=7.5Hz, 2H, CH₂), 4.32 (s, 2H, CH₂OH), 5.12 (s, 2H, NCH₂), 6.74 (d, J=8.1 Hz, 2H, ArH), 6.84 (s, 1H, CH═), 6.93 (d, J=7.2 Hz, 6H, ArH), 7.08 (d, J=8.1 Hz, 2H, ArH), 7.23-7.36 (m, 10H, ArH), 7.44-7.48 (m, 2H, ArH), 7.90-7.93 (m, 1H, ArH); ¹³C NMR (CDCl₃, 75 MHz): δ 13.7, 22.3, 26.7, 29.7, 46.3, 54.4, 82.8, 125.2, 126.2, 126.6, 127.6, 128.2, 129.7, 130.2, 130.7, 131.0, 135.3, 140.5, 141.2, 141.4, 150.1, 163.9; MS (FAB): M⁺=630, found: 631 (M⁺+1), 653 (M⁺+Na); IR (film, cm⁻¹) νₘₐₓ=3060, 2956, 2927, 2865, 1493, 1464, 1448, 1355, 1272, 1189, 1153, 1026, 906, 880, 822, 753, 699, 636.

### Preparation of Compound 6

Compound 5 (6.3 g, 10 mmol) was dissolved in a solvent mixture of 40 ml CH₂Cl₂ and water (1:1). After addition of CeCl₃.7H₂O (7.44 g, 20 mmol) and further 2 minutes of stirring, 10% aqueous solution of NaClO (37 ml) was added dropwise. Thereafter, stirring was continued for ten minutes, and a saturated aqueous solution of Na2SO₃ was added. The reaction mixture was extracted with CHCl₃, the organic phases were concentrated, and the obtained solid was purified by column chromatography, which led to compound 6 (4.65 g). The yield was 70%.

¹H NMR (CDCl₃, 300 MHz): δ 0.86 (t, J=7.2 Hz, 3H, CH₃), 1.23-1.33 (m, 2H, CH₂), 1.58-1.69 (m, 2H, CH₂), 2.49 (t, J=7.8Hz, 2H, CH₂), 3.30 (s, br, 1H, OH), 4.32 (s, 2H, CH₂OH), 5.14 (s, 2H, NCH₂), 6.78 (d, J=7.8 Hz, 2H, ArH), 6.94 (d, J=7.5 Hz, 6H, ArH), 7.12 (d, J=7.8 Hz, 2H, ArH), 7.23-7.37 (m, 10H, ArH), 7.43-7.51 (m, 2H, ArH), 7.94-7.97 (m, 1H, ArH); ¹³C NMR (CDCl₃, 75 MHz): δ 13.6, 22.3, 26.5, 29.5, 47.0, 52.7, 82.8, 124.9, 125.2, 126.1, 126.8, 127.5, 128.2, 129.7, 130.1, 130.6, 134.5, 140.7, 141.1, 141.2, 148.3, 163.8; MS (FAB): M⁺=664, found: 665 (M⁺+1), 687 (M⁺+Na); IR (film, cm⁻¹) νₘₐₓ=3184, 3061, 2958, 2931, 2869, 2244, 1577, 1492, 1466, 1447, 1356, 1255, 1189, 1160, 1078, 1028, 1005, 909, 881, 756, 733, 701, 640.

### Preparation of Compound 7

Compound 6 (6.64 g, 10 mmol) was dissolved in 20 ml THF and added with 20 ml 2N HCl. The reaction mixture was stirred at room temperature for four hours and then diluted with CHCl₃, washed with water and dried. The organic phases were concentrated, and the obtained solid was purified by column chromatography, which led to compound 7 (3.8 g). The yield was 90%.

¹H NMR (d-DMSO, 300 MHz): δ 0.78 (t, J=7.2 Hz, 3H, CH₃), 1.18-1.25 (m, 2H, CH₂), 1.41-1.46 (m, 2H, CH₂), 2.44 (t, J=7.5 Hz, 2H, CH₂), 4.32 (s, 2H, CH₂OH), 5.23 (s, 2H, NCH₂), 7.01 (d, J=8.1 Hz, 2H, ArH), 7.07 (d, J=8.1 Hz, 2H, ArH), 7.49-7.58 (m, 2H, ArH), 7.63-7.65 (m, 2H, ArH); ¹³C NMR (d-DMSO, 75 MHz): δ 13.5, 21.5, 25.7, 28.9, 46.4, 51.3, 123.5, 125.2, 125.6, 126.2 (2C), 127.7, 129.1 (2C), 130.5 (2C), 131.0, 136.1, 138.4, 141.0, 147.3, 155.0; MS (FAB): M⁺=422, found: 423 (M⁺+1), 445 (M⁺+Na); IR (film, cm⁻¹) νₘₐₓ=3351, 2959, 2932, 2870, 1936, 1709, 1575, 1464, 1422, 1361, 1257, 1226, 1078, 1007, 824, 758.

### Preparation of Compound 11

Compound 9 (9.5 g, 30 mmol) was dissolved in a mixture of 100 ml ethanol and 30 ml water. Thereafter, hydrazine hydrate (9 ml) was added, and the mixture was heated to reflux. After about one hour, a white solid separated, and after further nine hours of stirring under reflux, the mixture was cooled to room temperature. An NaOH solution (4.88 M, 100 ml) was added, and the reaction mixture was extracted with diethyl ether. The organic phase was dried over MgSO₄ and concentrated, which led to compound 11 (5 g). The yield was 90%.

¹H NMR (CDCl₃, 300 MHz): δ 1.36 (s, 2H, NH₂), 3.75 (s, 2H, NCH₂), 7.12 (d, J=8.1 Hz, 2H, ArH), 7.38 (d, J=8.1 Hz, 2H, ArH); ¹³C NMR (CDCl₃, 75 MHz): δ 45.5, 120.2, 128.6 (2C), 131.2 (2C), 142.0; IR (film, cm⁻¹) νₘₐₓ=3380, 2924, 2854, 2645, 2210, 1653, 1562, 1529, 1481, 1441, 1410, 1380, 1332, 1072, 1007, 905, 812, 789, 645, 618.

### Preparation of Compound 15

Compound 2 (6.8 g, 1.5 equivalents, 45 mmol) was dissolved in a mixture of 60 ml absolute ethanol and NEt₃ (12.5 ml, 3 equivalents) at 0° C., and then compound 11 (5.6 g, 1 equivalent, 30 mmol) was added. The reaction mixture was stirred at room temperature for ten hours and then K₂CO₃ (6.2 g, 45 mmol, 1.5 equivalents) and compound 8 (6.9 g, 1.3 equivalents, 36 mmol) were added, and stirring was continued for twelve hours at room temperature. Thereafter, extraction with CHCl₃ was carried out, the organic phase was concentrated, and the radical was purified by column chromatography, which led to compound 15 (3.74 g). The yield was 39%.

¹H NMR (CDCl₃, 300 MHz): δ 0.88 (t, J=7.2 Hz, 3H, CH₃), 1.31-1.38 (m, 2H, CH₂), 1.63-1.71 (m, 2H, CH₂), 2.63 (t, J=8.1 Hz, 2H, CH₂), 5.50 (s, 2H, NCH₂), 6.88 (d, J=8.4 Hz, 2H, ArH), 7.42 (d, J=8.4 Hz, 2H, ArH), 7.77 (s, 1H, CH═), 9.64 (s, 1H, CHO); ¹³C NMR (CDCl₃, 75 MHz): δ 13.6, 22.4, 26.4, 29.3, 47.5, 121.7, 128.0 (2C), 131.2, 131.9 (2C), 135.2, 143.7, 156.6, 178.7; MS (FAB): M⁺=320, found: 321 (M⁺+1), 323 (M⁺+3); IR (film, cm⁻¹) νₘₐₓ=2958, 2932, 2868, 1671, 1533, 1485, 1463, 1407, 1373, 1161, 1072, 1011, 813, 769, 648.

### Preparation of Compound 16

A solution of p-bromo toluene (17 g, 100 mmol) in 100 ml of dried THF was added dropwise to a mixture of magnesium powder (3.6 g, 150 mmol), iodine (200 mg) and 4 drops of 1,2-dibromo ethane within one hour. Thereafter, heating for one hour at reflux and then cooling to −78° C. were carried out, and trimethyl borate (10.3 g, 10 mmol) was added. The reaction mixture was stirred for another two hours, and then quenched by addition of water. After extraction with ethyl acetate the organic phase was washed with water, dried and concentrated. The radical was purified by column chromatography, which led to compound 16 (10.2 g) as colourless crystals. The yield was 75%.

¹H NMR (CDCl₃, 300 MHz): δ 2.41 (s, 3H, CH₃), 7.28 (d, J=7.5 Hz, 2H, ArH), 8.09 (d, J=7.5 Hz, 2H, ArH); ¹³C NMR (CDCl₃, 75 MHz): δ 21.9, 128.8 (2C), 135.7 (3C), 142.9; MS (EI): m/z(%): 354 (M⁺, 100), 262 (19), 193 (17), 145 (18), 119 (36), 91 (39), 43 (47); IR (film, cm⁻¹) νₘₐₓ=3045, 3022, 2918, 1920, 1613, 1517, 1406, 1367, 1347, 1307, 1179, 1109, 1081, 818, 736, 685, 528, 477.

### Preparation of Compound 17

Trimerised boric acid 16 (5 g, 14.1 mmol), pinacole hexahydrate (11.5 g, 50.8 mmol) were dissolved in 100 ml of cyclohexane and the solution was refluxed for ten hours to remove water. Thereafter, the cyclohexane was removed by distillation under reduced pressure, and the radical was purified by column chromatography, which led to compound 17 (7.8 g) as an oil. The yield was 84%.

¹H NMR (CDCl₃, 300 MHz): δ 1.37 (s, 12H, 4CH₃), 2.40 (s, 3H, CH₃), 7.22 (d, J=7.5 Hz, 2H, Ar), 7.75 (d, J=7.5 Hz, 2H, Ar); ¹³C NMR (CDCl₃, 75 MHz): δ 21.7, 24.8 (4C), 83.5 (2C), 128.5 (2C), 134.8 (3C), 141.3; MS (EI): m/z (%): 218 (M⁺, 23), 203 (33), 132 (52), 119 (100), 91 (22), 43 (58); IR (film, cm⁻¹) νₘₐₓ=3046, 2979, 2928, 1613, 1519, 1448, 1398, 1361, 1320, 1268, 1214, 1146, 1089, 1023, 962, 859, 816, 726, 656.

### Preparation of Compound 18

Compound 17 (5 g, 22.9 mmol), NBS (5.3 g, 29.8 mmol) and AIBN (200 mg) were dissolved in 40 ml cyclohexane and the solution was heated to reflux for 5.5 hours. Thereafter, filtration was carried out under reduced pressure, and the filtrate was concentrated. The radical was purified by column chromatography, which led to compound 17 (5.86 g) as an oil. The yield was 86%.

¹H NMR (CDCl₃, 300 MHz): δ 1.35 (s, 12H, 4CH₃), 4.45 (s, 2H, CH₂), 7.40 (d, J=7.2 Hz, 2H, ArH), 7.81 (d, J=7.2 Hz, 2H, ArH); ¹³C NMR (CDCl₃, 75 MHz): δ 24.8 (4C), 33.2, 83.8 (2C), 125.6, 128.2 (2C), 135.2 (2C), 140.6; MS (EI): m/z(%): 297 (M⁺+1, 5), 295 (M⁺−1, 5), 281 (3), 283 (3), 217 (100), 197 (15), 131 (13), 117 (50), 91 (12), 43 (39); IR (film, cm⁻¹) νₘₐₓ=3044, 2974, 2919, 1937, 1609, 1512, 1396, 1356, 1320, 1269, 1217, 1143, 1085, 1017, 960, 845, 784, 655, 602.

### Preparation of Compound 19

Compound 18 (16 g, 53.9 mmol), phthalimide (10.3 g, 72 mmol) and K₂CO₃ (9.7 g, 72 mmol) were heated for twelve hours under reflux in 60 ml of dry acetone. Thereafter, the acetone was removed by distillation under reduced pressure, and 100 ml of water were added to dissolve the inorganic salts. The reaction mixture was filtered under reduced pressure, washed with water and dried, which led to compound 19 (17.6 g) as a white solid. The yield was 90%.

¹H NMR (CDCl₃, 300 MHz): δ 1.31 (s, 12H, 4CH₃), 4.85 (s, 2H, CH₂), 7.42 (d, J=6.6 Hz, 2H, ArH), 7.67-7.70 (m, 2H, ArH), 7.76 (d, J=6.6 Hz, 2H, ArH), 7.81-7.84 (m, 2H, ArH); ¹³C NMR (CDCl₃, 75 MHz): δ 24.8 (4C), 41.6, 83.7 (2C), 123.3 (2C), 127.8 (2C), 132.1, 133.9 (2C), 135.1 (3C), 139.3 (2C), 167.9 (2C); MS (EI): m/z (%): 363 (M⁺, 100), 348 (17), 264 (37), 217 (34), 160 (31), 130 (29), 117 (92), 91 (16), 76 (36), 43 (78); IR (film, cm⁻¹) νₘₐₓ=2989, 2941, 1772, 1718, 1610, 1429, 1393, 1358, 1342, 1141, 1087, 1020, 962, 938, 856, 786, 718, 659.

### Preparation of Compound 21

Compound 19 (2.5 g, 6.9 mmol) and hydrazine hydrate (0.47 ml, 80%, 7 mmol) were dissolved at room temperature in 30 ml of methanol, and the mixture was heated under reflux. After twelve hours, the mixture was cooled to room temperature and filtered under reduced pressure. The white solid was disposed of, and the filtrate was concentrated to dryness. Water was removed under a protecting gas atmosphere by using dry benzene. Then NEt₃ (2.9 ml), dry methanol (10 ml) and compound 2 (2.1 g, 14 mmol) were added and the reaction mixture was stirred at room temperature for ten hours. Thereafter, K₂CO₃ (952 mg, 7 mmol) and compound 8 (1.6 g, 8.3 mmol) were added and stirring was continued for ten hours at room temperature. Thereafter, some water was added to the reaction mixture, and an extraction with CHCl₃ was carried out. Washing with water was carried out, and the organic phase was separated, dried and concentrated. The radical was purified by column chromatography, which led to compound 21 (85 mg) as an oil. The yield was 4%.

¹H NMR (CDCl₃, 300 MHz): δ 0.88 (t, J=7.5 Hz, 3H, CH₃), 1.25-1.38 (m, 14H, CH₂ und 4CH₃), 1.63-1.73 (m, 2H, CH₂), 2.62 (t, J=7.5 Hz, 2H, CH₂), 5.59 (s, 2H, ArCH₂), 6.99 (d, J=7.8 Hz, 2H, ArH), 7.73 (d, J=7.8 Hz, 2H, ArH), 7.78 (s, 1H, NCH═), 9.66 (s, 1H, CHO); ¹³C NMR (CDCl₃, 75 MHz): δ 13.7, 22.4, 24.8(4C), 26.5, 29.3, 48.2, 83.9(2C), 125.5(2C), 131.4, 135.3 (3C), 139.2, 143.6, 156.8, 178.7; MS (FAB): M⁺=368, found: 369 (M⁺+1); IR (film, cm⁻¹) νₘₐₓ=3044, 2975, 2933, 2870, 1671, 1614, 1533, 1464, 1405, 1362, 1326, 1270, 1160, 1145, 1089, 1021, 963, 858, 821, 793, 721, 654.

### Preparation of Compounds 22 and 23

A suspension of o-bromo benzonitrile (9.1 g, 50 mmol), NH₄Cl (3.5 g, 65 mmol), NaN₃ (4.3 g, 65 mmol) and LiCl in 80 ml DMF was heated at 100° C. and stirred for twelve hours. A large proportion of the solvent was removed by distillation at 120° C. under reduced pressure. The radical was made alkaline by using a 10% aqueous solution of NaOH until a pH of 12 was reached. The reaction mixture was extracted with ethyl acetate, and the inorganic phase was acidified with concentrated HCl up to a pH value of 2 which led to the separation of a white solid. This solid was filtered off under reduced pressure using a Buchner funnel, washed with water and dried, which led to compound 22 (10 g, yield 90%). Compound 22 was dissolved in 30 ml CH₂Cl₂. The mixture was cooled in an ice water bath to 0° C., and NEt₃ (8 ml) was added. Thereafter, Ph₃CCl (13.2 g, 47 mmol) was added in 3 portions within ten minutes, and the reaction mixture was heated to room temperature. After three hours of stirring, filtration with a Buchner funnel under reduced pressure was carried out. Washing with water was carried out and drying, which led to compound 23 (18.9 g). The yield was 90%.

¹H NMR (CDCl₃, 300 MHz): δ 7.18-7.36 (m ,17H, ArH), 7.66 (d, J=7.8 Hz, 1H, ArH), 7.88 (d, J=7.8 Hz, 1H, ArH); ¹³C NMR (CDCl₃, 75 MHz): δ 83.3, 122.2, 127.3, 127.7, 128.3, 128.7, 130.3, 131.1, 131.6, 133.9, 141.2, 162.9.

### Preparation of Compound 12

Benzonitrile (10.3 g, 100 mmol), NH₄Cl (6.9 g, 1.3 equivalents), NaN₃ (8.5 g, 1.3 equivalents) and LiCl (300 mg) were dissolved in 100 ml of DMF, and the reaction mixture was stirred at 100° C. Thereafter, a large proportion of the solvent was removed under reduced pressure. The radical was made alkaline with 10% aqueous NaOH until a pH of 12 was reached. After extraction with ethyl acetate the aqueous phase was separated and acidified with concentrated hydrochloric acid until a pH of 2 was reached. The precipitate was filtered off with a Büchner funnel, washed with water and dried, which led to 5-phenyl tetrazole (13.5 g, melting point 208-209° C.). The yield was 96%.

¹H NMR (d-DMSO, 300 MHz) δ 7.55-7.57 (3H, m), 8.01-8.03 (2H, m); ¹³C NMR (d-DMSO, 75 MHz) δ 129.5, 132.4, 134.8, 136.7, 160.7; MS (EI) m/z (%): 146 (M+, 42), 118 (100), 103 (17), 91 (46), 77 (32), 63 (48); IR (film, cm⁻¹) νₘₐₓ=3055, 2982, 2837, 2607, 2545, 1607, 1562, 1485, 11463, 1409, 1163, 1056, 1013, 725, 703, 686.

5-phenyl tetrazole (6.6 g, 45 mmol) was dissolved in 20 ml of CH₂Cl₂, and added with NEt₃ (8 ml, 1.3 equivalents). The reaction mixture was cooled in an iced water bath to 0° C. and Ph₃CCl (13.2 g, 1.05 equivalents) was added within 10 minutes in three portions. Thereafter, the mixture was warmed to room temperature and stirred for three hours. The reaction mixture was filtered, washed with water and dried, to obtain compound 12 (16.5 g, melting point 163-164° C.). The yield was 94%.

¹H NMR (CDCl₃, 300 MHz) δ 7.21-7.24 (6H, m), 7.37-7.39 (9H, m), 7.47-7.49 (3H, m), 8.19-8.20 (2H, m); ¹³C NMR (CDCl₃, 75 MHz) δ 83.0, 127.0, 127.5, 127.7, 128.3, 128.7, 130.3, 141.3, 164.0; IR (film, cm⁻¹) νₘₐₓ=3058, 1490, 1465, 1445, 1186, 1028, 874, 763, 748, 697, 635.

### Preparation of Compound 13

A solution of compound 12 (10 g, 25.8 mmol) in THF (30 ml) was cooled under an argon atmosphere to −20° C. Thereafter, BuLi (1 M, 27 ml, 1.05 equivalents) was added. The temperature was raised to −5° C., and stirring was carried out for one hour. In the meantime, a large amount of solid precipitated. It was cooled again to −25° C., and B(OMe)₃ (4.3 ml, 1.5 equivalents) was slowly added via a syringe. Thereafter, the reaction mixture was allowed to warm to 20° C. and stirred for half an hour. The solvent was reduced under reduced pressure to ⅓ of the original amount which led to the formation of a white solid. The solid was filtered off, washed with 20% THF in H₂O (40 ml) and water (40 ml) and dried, which led to compound 13 (10.4 g). The yield was 94%. Compound 13 can be further used without purification.

1. Process for preparing a compound of the general formula I 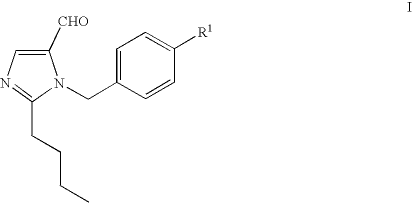
in which R¹ is an R^{1a} radical or an R^{1b} radical, where
R^{1a} is a radical of the general formula II 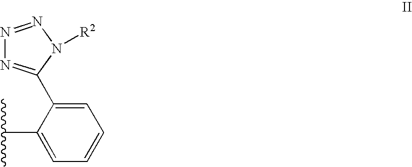
in which R² is a tetrazole protecting group, or
R^{1b} is a radical which is suitable to couple the phenylene group of the compound of the general formula I by reaction with an R³ radical which is complementary thereto and is part of a compound of the general formula III
R³—R⁴ III
which contains another phenylene unit and in which R⁴ is a radical of the general formula II to form a C—C bond between the phenylene group of the compound of the general formula I and the phenylene group of the compound of the general formula III, by reacting a compound of the general formula IV 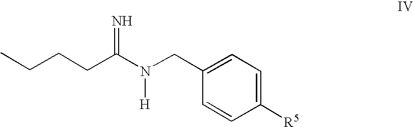
in which R⁵
in the case that R¹ in formula I is an R^{1a} radical, is a radical of the general formula II, and
in the case that R¹ in the formula I is an R^{1b} radical, is as defined for the R^{1b} radical in formula I
with a compound of the general formula V 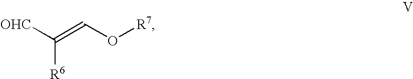
in which R⁶ is a halogen from the group of Cl, Br, I, preferably Br, and R⁷ is a branched or unbranched C₁-C₆-alkyl group, preferably an isopropyl group.

2. Process according to claim 1, wherein the tetrazole protecting group R² in formula II is triphenylmethyl or tert-butyl.

3. Process according to claim 1, wherein the reaction is performed in the presence of a weak Brønsted base.

4. Process according to claim 1, wherein the R^{1b} radical of the compound of the general formula I or R⁵ radical in the compound of the general formula IV is a radical which is capable of reacting with the R³ radical in a Suzuki, Stille or Grignard reaction.

5. Process according to claim 1, wherein the R^{1b} radical in the compound of the general formula I or R⁵ radical in the compound of the general formula IV is defined as follows:
halogen,
a radical of the general formula VI 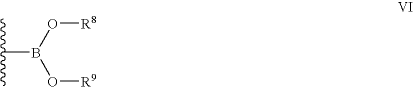
in which R⁸ and R⁹ are each hydrogen, a C₁- to C₆-alkyl group or together are a C₁- to C₆-alkanediyl group,
a trialkyltin radical or
when a compound of the general formula I with the R^{1b} radical is used in the process, a magnesium(II) halide radical,
and where,
when R^{1b} or R⁵ is a halogen, R³ is a radical of the general formula VI, a trialkyltin radical or, when a compound of the general formula I with the R^{1b} radical is used in the process, a magnesium(II) halide radical and vice versa.

6. Compound of the general formula IV as defined in claim 1 in which R⁵ is a radical of the general formula II.

7. Process for preparing the compound of the general formula IV in which R⁵ is a radical of the general formula II, in which a compound of the general formula VII 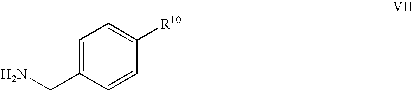
in which R¹⁰ is a radical of the formula II is reacted with a compound of the general formula VIII 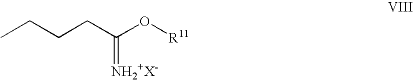
in which R¹¹ is a C₁- to C₁₂-alkyl radical and X⁻ is the anion of a mineral acid, in the presence of a Brønsted base.

8. Process according to claim 7, wherein the compound of the formula VII is provided by
I. providing a compound of the general formula IX 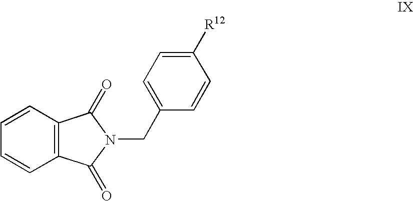
in which R¹² is a radical of the general formula II and
II. preparing the compound of the general formula VII from the compound of the general formula IX under conditions as are typical for a Gabriel reaction.

9. Compound of the general formula I as defined in claim 1 in which R¹ is bromine.

10. Compound of the general formula IV as defined in claim 1 in which R⁵ is halogen, especially bromine.

11. Process according to claim 1, wherein the R^{1b} radical in formula I or the R⁵ radical in formula IV is bromine.

12. Process for preparing a compound of the general formula IV in which R⁵ is halogen, wherein a benzylamine derivative para-substituted by a halogen atom is reacted with a compound of the general formula VIII in the presence of a Brønsted base.

13. Process according to claim 12, wherein the compound of the general formula IV in which R⁵ is halogen is provided by preparing a benzylamine derivative para-substituted by a halogen atom in a Gabriel reaction with phthalimide from a benzyl halide para-substituted by a halogen atom.

14. Process for preparing a compound of the general formula I with an R^{1a} radical, by reacting a compound of the general formula IV in which R⁵ is halogen with a compound of the general formula III in which R³ is a radical of the general formula VI, a trialkyltin radical or a magnesium(II) halide radical, under conditions as are typical for a Suzuki, Stille or Grignard reaction.

15. Compound of the general formula I as defined in claim 1 in which R¹ is a radical of the general formula VI, a trialkyltin radical or magnesium(II) halide radical.

16. Compound of the general formula IV as defined in claim 1 in which R⁵ is a radical of the general formula VI, a trialkyltin radical or magnesium(II) halide radical.

17. Process according to claim 5, in which R⁸ and R⁹ in formula VI together are 2,3-dimethylbutane-2,3-diyl.

18. Process for preparing a compound of the general formula IV in which R⁵ is a radical of the general formula VI, by reacting a benzylamine derivative para-substituted by an R⁵ radical of the general formula VI with a compound of the general formula VIII in the presence of a Brønsted base.

19. Process according to claim 18, wherein a compound of the general formula IV in which R⁵ is a radical of the general formula VI is provided by preparing a benzylamine derivative para-substituted by an R⁵ radical of the general formula VI in a Gabriel reaction with phthalimide from a benzyl halide para-substituted by an R⁵ radical of the general formula VI.

20. Process for preparing a compound of the general formula I in which R¹ is a radical of the general formula II, by reacting a compound of the general formula IV in which R⁵ is a radical of the general formula VI with a compound of the general formula III in which R³ is halogen under conditions as are typical for a Suzuki reaction.

21. Compound of the general formula XI 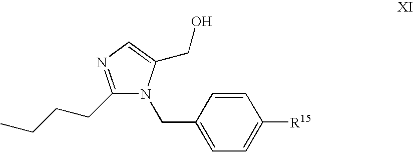
in which R¹⁵ is a radical of the general formula II.

22. Process for preparing losartan or one of its pharmacologically acceptable salts according to claim 1, by
a) in a step (a) proceeding from a compound of the general formula I with an R^{1a} radical, preparing the compound of the general formula XI by reducing the formyl group with which the imidazole group is substituted in a customary manner to a hydroxymethyl group,
b) in a step (b), replacing the sole hydrogen atom remaining in the imidazole group of the compound prepared in step (a) with chlorine in a customary manner and
c) in a step (c), eliminating the tetrazole protecting group and optionally
d) from losartan, preparing one of its pharmacologically acceptable salts.

23. Process according to claim 1, by using one or more catalysts comprising one or more transition metals, preferably selected from MnCl₂, CrCl₃, FeCl₂, Fe(acac)₃, FeCl₃, Fe(salen)Cl, NiCl₂(PPh₃)₂, COCl₂(dppe), COCl₂(dpph), Co(acac)₂, COCl₂(dppb), PdCl₂(PPh₃)₂ or Pd(PPh₃)₄.

24. Process for preparing an imidazole derivative substituted by chlorine at one or more carbon atoms of the imidazole ring (imidazole derivative A), by reacting imidazole or an imidazole derivative which bears a hydrogen atom at at least one carbon atom of the imidazole ring (imidazole derivative B) with CeCl₃ and an alkali metal salt of a hypohalic acid to prepare a losartan derivative in which the hydrogen atom of the tetrazole group has been replaced by a tetrazole protecting group, and wherein the imidazole derivative (B) used is the compound of the general formula XI.

25. Process according to claim 24, wherein the imidazole derivative (A) prepared is a compound which is substituted by chlorine on the carbon atom of the imidazole ring in the 4 or 5 position or at both aforementioned positions, and wherein the imidazole derivative (B) used is a compound which also bears a hydrogen atom on the carbon atom of the imidazole ring in the 4 or 5 position or at both aforementioned positions.

26. Process according to claim 24, wherein the CeCl₃ and the alkali metal salt of a hypohalic acid are used in stoichiometric amounts or in excess.

27. Process according to claim 24, wherein the alkali metal salt of the hypohalic acid used is a potassium or sodium salt.

28. Process according to claim 24, wherein the alkali metal salt of the hypohalic acid used is an alkali metal salt of hypochlorous acid.

29. Process according to claim 24, wherein the reaction is performed in a 2-phase system in which one phase is formed from an aqueous solution and the other phase from a solution which comprises an organic solvent which does not have unlimited miscibility with water.

30. Process according to claim 22, wherein the sole hydrogen atom still remaining in the imidazole group of the compound prepared in step (a) is replaced in step (b) by chlorine in a customary manner, by reacting the compound prepared in step (a) with CeCl₃ and an alkali metal salt of a hypohalic acid as reagents.
